(19) United States
(12) Patent Application Publication
(10) Pub. No.: US 2023/0355685 A1
(43) Pub. Date: Nov. 9, 2023

(54) RAW MATERIAL PRODUCTION FROM STICHOPUS VARIEGATUS

(71) Applicant: Sucipto KOKADIR, Jarkata Barat (ID)

(72) Inventor: Sucipto KOKADIR, Jarkata Barat (ID)

(21) Appl. No.: 17/771,416

(22) PCT Filed: Jun. 22, 2020
(86) PCT No.: PCT/IB2020/055885

§ 371 Date: Apr. 22, 2022

(30) Foreign Application Priority Data
Aug. 29, 2019 (ID) P00201907575
Feb. 25, 2020 (ID) S00202001562
May 12, 2020 (ID) S00202003434

(51) Int. Cl. A61K 35/616; A23L 17/00; B01D 61/14; A23L 29/30; A23K 10/14; A23K 10/20; A23K 20/163; A61K 8/98; A01K 61/90; A22B 5/00; A22C 17/00

(52) U.S. Cl. CPC A61K 35/616; A23L 17/65; B01D 61/145; A23L 29/35; A23K 10/14; A23K 10/20; A23K 20/163; A61K 8/987; A01K 61/90; A22B 5/0005; A22C 17/0026; A22B 5/0082; A61K 2236/13; A61K 2236/15; A61K 2236/19; A61K 2236/31; A61K 2236/51

(57) ABSTRACT

A method of producing a bioproduct from a sea cucumber wherein the steps of weighing the sea cucumbers, cleaning the sea cucumber, and milling the sea cucumber in a milling machine for creating a sea cucumber pulp may be performed. Further, the processing of the sea cucumber pulp in a multi-step hydrolysis membrane reactor may be completed for the formation of an unfiltered bioproduct. Such a product may need filtering through an ultrafiltration membrane system to obtain a filtered bioproduct. Followed by inactivation of the protease to create a filtered bioproduct, to which drying may then be needed. The final step may be the application of the filtered bioproduct to a relevant industrial use may be described herein.

### RELATED APPLICATIONS

This application claims the benefit of Indonesian patent number P00201907575, filed Aug. 29, 2019; Indonesian patent number S00202003434, filed May 12, 2020; and Indonesian patent number S00202001562, filed, Feb. 25, 2020, the contents of each of which are incorporated by this reference in their entireties for all purposes as if fully set forth herein.

### TECHNICAL FIELD

The disclosure herein relates generally to processes and methods for producing bioproduct from a sea cucumber and generally utilizing the sea cucumber as a natural source for biologically useful raw materials. More particularly, this disclosure herein relates to the selecting, handling, processing, and drying of the sea cucumber as a natural source to produce raw materials for use in the pharmaceutical, cosmetic, food and beverage, and animal feed industries by way of hydrolyzing said sea cucumber with a protease.

### BACKGROUND

Sea cucumbers, also known as sandfish, are a species of the phylum Echinodermata, meaning that they are spinney skinned, under the class Holothuroidea. Generally speaking, they are marine animals and are distributed in oceans the world over. They are generally found near living coral, rocks, or seaweeds in warm shallow waters. They are also found extensively in benthic environments. It is estimated that at oceanic depths beyond 8.9 kilometers, they can comprise approximately 90% of the total mass of microfauna.

Sea cucumbers are one of the most easily recognized marine biota groups consisting of approximately 1,250 species, with new species being identified in the Indo-Pacific Ocean and globally routinely. The body shape of the sea cucumber is generally cylindrical, extending from the tip of the mouth toward the anus (orally to aborally). Sea cucumbers may be further characterized by a soft and elastic body and having varied shapes, such as rounded, cylindrical, rectangular, or elongated round like a snake. The sea cucumber body length varies by species and age and may range from 2 cm to 150 cm.

Because Indonesia is an island nation, many sea cucumbers may be readily sourced from the national marine waters. Traditionally, sea cucumbers are used as a food resource with the earliest known record of human consumption dating back to the 18th century. The sea cucumbers may be consumed either fresh or after drying. Many cultures in Eastern, and Southeastern Asian consider the sea cucumber to be a delicacy, including Indonesia. Nutritionally, sea cucumbers have an impressive profile of valuable nutrients such as Vitamin A, Vitamin B1 (thiamine), Vitamin B2 (riboflavin), Vitamin B3 (niacin), and minerals, especially calcium, magnesium, iron, and zinc.

Accordingly, Indonesia is one of the largest commercial export nations for sea cucumber among the countries which export sea cucumber in the world. The distribution of sea cucumbers in Indonesia includes the waters off the coast of Sumatra, the north coast of Java, Bali, Nusa Tenggara, and is also widespread in eastern Indonesia. The marine biota of the national Indonesian waters has been widely studied and show to be capable of producing a wide variety of bioactive peptides produced by enzymatic hydrolysis. The bioactive peptides have been identified from sources including both directly from the marine biota and downstream from processing waste. These various bioactive peptides have been reported to have a physiological function which may include antioxidant properties, antimicrobial properties, and potential antihypertensive agents. The bioactivity of these various peptides has been shown in vitro as well in vivo and the relationship between the two has been examined as well. However, the investigation of various marine biota is ongoing, and sea cucumbers are a natural extension of this investigation.

Sea cucumbers are also known to have bioactive compounds including lectins, sterols, glycosides, chondroitin sulfate, vitamins, amino acids, glucosamine, minerals, mucopolysaccharides, collagen, and they have a protein content of more than 80%. This makes the sea cucumber a viable target for the development of healthy food products, supplements, cosmetic ingredients, animal feed supplements, pharmaceuticals, and naturopathic medicines.

Because sea cucumbers are able to offer such a wide array of bioactive compounds, there exists a need for specific species of sea cucumber to undergo the steps of research to further utilize and exploit the various active ingredients offered by sea cucumbers. Said need may cause innovative steps to be directed towards specific species of sea cucumber to make and use sea cucumber powder hydrolysate products. Such hydrolysis processes may be used as a cornerstone for the production of other raw materials and preparations for the pharmaceutical industry, cosmetics industry, food and beverage industry, animal feed industry, and other industries and their use.

### SUMMARY

Certain deficiencies of the prior art are overcome by the provision of embodiments of methods and systems in accordance with the present disclosure. Herein disclosed may be a method of producing a hydrolysate bioproduct from a sea cucumber wherein the steps of: weighing the sea cucumbers, cleaning the sea cucumber, milling the sea cucumber in a milling machine for creating a sea cucumber pulp, processing the sea cucumber pulp in a multi-step hydrolysis membrane reactor for creating a hydrolyzed sea cucumber, filtering the hydrolyzed sea cucumber through an ultrafiltration membrane system to obtain a purified hydrolysate bioproduct, inactivating the purified hydrolysate bioproduct to inactivate the hydrolysis enzyme, drying the hydrolysate bioproduct, and applying the hydrolysate bioproduct to a relevant industrial use may be described.

Accordingly, the method herein described of producing a hydrolysate bioproduct from a sea cucumber may involve individual sea cucumbers greater than a predetermined weight in grams and maybe are discarded if not of sufficient weight due to sustainability concerns. Further, the cleaning of the sea cucumber may be achieved by splitting open the sea cucumber with a cutting tool from mouth to anus and subsequently removing bowels by rinsing with water. The sea cucumber pulp may be assessed for proper particulate size and consistency. Deionized water may be utilized and combined with the sea cucumber pulp to form an aqueous mixture. The aqueous mixture may constitute a percentage of the deionized water of approximately 60% and a percentage of the sea cucumber pulp of approximately 40%. The aqueous mixture may be added to a multi-step hydrolysis membrane reactor. The multi-step hydrolysis membrane reactor may be operated at a temperature between 45 degrees centigrade to 65 degrees centigrade.

Because of possible variations in pH of any given solution, a step in the process herein disclosed may include a check of the pH of the aqueous mixture and an adjustment if needed to a range between approximately 6.5 to 7.5. A protease may be added to the multi-step hydrolysis membrane reactor to catabolize proteins from the aqueous mixture. The aqueous mixture may remain in the multi-step hydrolysis membrane reactor for a measured time between 2 to 5 hours. The protease in the aqueous mixture may be inactivated by increasing the temperature of the aqueous mixture to between 85 degrees centigrade to 95 degrees centigrade after the measured time is complete. The purified hydrolysate bioproduct may be dried by using a spray dry machine. The spray dry machine may use a maltodextrin DE 10-12 filler. The maltodextrin DE 10-12 filler may constitute between 5% and 30% of a final bioproduct. The relevant industry may be a pharmaceutical industry, a cosmetic industry, a food, and beverage industry, a livestock feed industry, or another industry not explicitly identified in this disclosure.

### BRIEF DESCRIPTION OF THE DRAWINGS

The foregoing and other features of the present disclosure will become more fully apparent from the following description, taken in conjunction with the accompanying drawings. Understanding that these drawings depict only several embodiments in accordance with the disclosure and are not to be considered limiting of its scope, the disclosure will be described with additional specificity and detail through the use of accompanying drawings. Accordingly, further advantages of the present disclosure may become apparent to those skilled in the art with the benefit of the following detailed description of the preferred embodiments and upon reference to the accompanying drawings in which:

FIG. 1 is a simplified flow diagram illustrating a method for obtaining a bioproduct from sea cucumber according to one non-limiting embodiment;

FIG. 2 is a simplified flow diagram illustrating a method for obtaining bioproduct from sea cucumber, wherein the process of hydrolysis is better illustrated, according to one non-limiting embodiment;

FIG. 3 is a simplified flow diagram illustrating a method for applicability of bioproduct obtained from sea cucumber according to one non-limiting embodiment;

FIG. 4 is a simplified flow diagram illustrating a method for obtaining a bioproduct from sea cucumber according to one non-limiting embodiment;

FIG. 5 is a simplified flow diagram illustrating a method for obtaining a bioproduct from sea cucumber according to one non-limiting embodiment;

FIG. 6 is a simplified flow diagram illustrating a method for obtaining a bioproduct from sea cucumber according to one non-limiting embodiment;

FIG. 7 is a simplified flow diagram illustrating a method for obtaining a bioproduct from sea cucumber according to one non-limiting embodiment; and,

FIGS. 8A and 8B are of a simplified flow diagram illustrating a method for obtaining a bioproduct from sea cucumber according to one non-limiting embodiment.

### DETAILED DESCRIPTION OF THE PREFERRED EMBODIMENTS

Embodiments of systems, components, and methods of assembly, use, and manufacture will now be described with reference to the accompanying figures. Although several embodiments, examples, and illustrations are disclosed below, it will be understood by those of ordinary skill in the art that the embodiments described herein extend beyond the specifically disclosed configurations, examples, and illustrations, and can include other users of the disclosure and obvious modifications and equivalents thereof. The terminology used in the descriptions presented herein is not intended to be interpreted in any limited or restrictive manner simply because it is being used in conjunction with a detailed description of certain specific embodiments of the disclosure. In addition, embodiments of the disclosure can comprise several novel features and no single feature is solely responsible for its desirable attributes or is essential to practicing any one of the several embodiments herein described.

Certain terminology may be used in the following description for the purpose of reference only, and thus are not intended to be limiting. For example, terms such as “above” and “below” refer to directions in the drawings to which reference is made. Terms such as “front,” “back,” “left,” “right,” “rear,” “top,” “bottom” and “side” describe the orientation and/or location of portions of the components or elements within a consistent but arbitrary frame of reference which is made clear by reference to the text and the associated drawings describing the components or elements under discussion. Moreover, terms such as “first,” “second,” “third,” and so on may be used to describe separate components. Such terminology may include the words specially mentioned above, derivatives thereof, and words of similar import.

Moreover, the term “sea cucumber” may refer herein to the species Stichopus variegatus or another species. The species Stichopus variegatus may belong to the Kingdom of Animalia and the Phylum of Echinodermata. Further, Stichopus variegatus may belong to the Class Holothuroidea, the Order Aspidochirotida, the Family Stichopodidae, and the genus Stichopus. This classification system may not be considered the final classification system as the discipline of taxonomy is subject to change as discoveries are made known. This classification system is what is currently known and understood for the species Stichopus variegatus at the time of this disclosure.

The term “benthic” or “benthic environment” shall refer herein to the lowest point of a column of water and may comprise a floor of said column of water. Such environments may comprise sediment, silt, sand, gravel, dirt, mud, or clay. Moreover, where such benthic regions may be found and located in marine environments, the term benthic shall not mean the lowest point of any one given ocean, but rather the lowest point beneath an imaginary line passing from the center of mass of Earth and extending toward the sky. Within this meaning, benthic and/or a benthic environment may be understood as existing under a few inches of water, or could also exist under a column of water one mile or deeper. However, it must be particularly stated the species Stichopus variegatus tend to exist at deeper benthic depths than other sea cucumbers. Because the species Stichopus variegatus do exist at deeper depths, professional divers are often needed in the harvesting phase of the various method embodiments herein described. Such professional divers often are required to utilize mix gas diving, for example, a mixture of oxygen, helium, and nitrogen.

The term “amount” may refer to a quantity of a thing, such quantity may be defined by a volumetric measure, percentage composition measure, weigh by mass measure, or other measures not explicitly stated herein. Accordingly, it is intended that the term “amount” should carry the broadest possible understanding of the term.

Referring to the drawings, like reference numerals designate identical or corresponding features throughout the several views. Described herein are certain non-limiting embodiments and methods of utilizing sea cucumber for pharmaceutical purposes, functional food purposes, cosmetic purposes, animal feed supplement purposes, and other industrial uses. The application of the terms start 102 and end 124 as depicted in the various figures refers solely to the beginning and terminal points of the flow charts, demarcating the flow charts in the various figures shall not imply that the flow charts are thereby constrained by a beginning and terminal point of the figures. It should be well understood by those familiar and practicing in the arts, that such flow charts are included as examples only and do not limit the various disclosures herein.

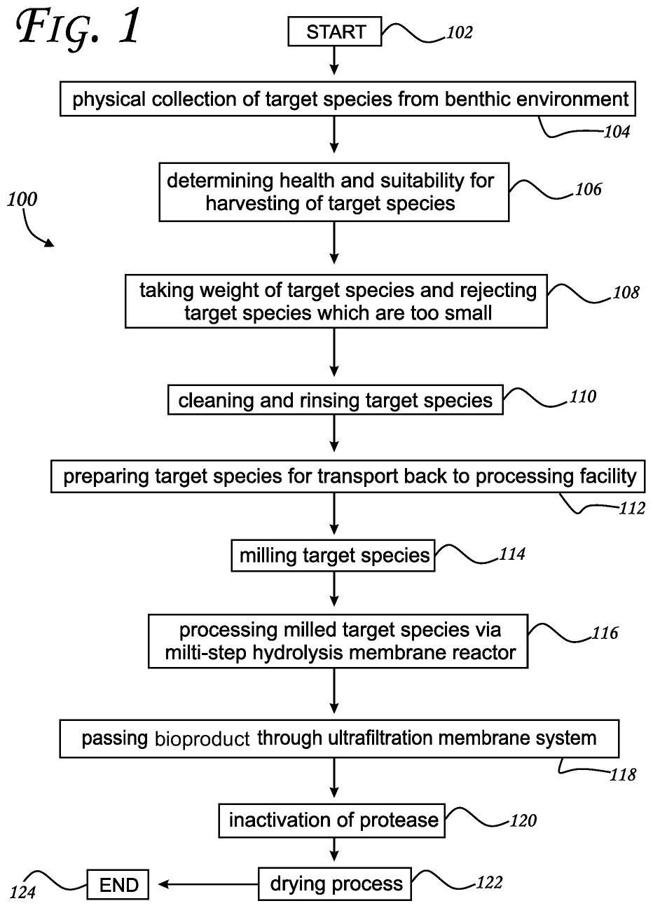

Accordingly, FIG. 1 illustrates a simple flow chart that may describe an industrial use 100 for sea cucumber, specifically the species Stichopus variegatus. Described in FIG. 1 after the start 102 of the flow chart may be a collection process 104 for sea cucumber, a pre-harvest assessment 106 processes for sea cucumber, a weight assessment 108, a cleaning process 110 of the sea cucumber, a step describing the transport 112 process to a processing facility (not shown), a milling process 116, an application of the milled sea cucumber to a multi-step hydrolysis membrane reactor 116, the use of an ultra-filtration membrane system 118 to purify an aqueous mixture, an inactivation process 120, and a drying process 122 before reaching the end 124 of the flow chart of FIG. 1. The steps illustrated in FIG. 1 are not exhaustive, nor exclusive of other steps, but represents only one of many potential embodiments for processing of the industrial use 100 for sea cucumber.

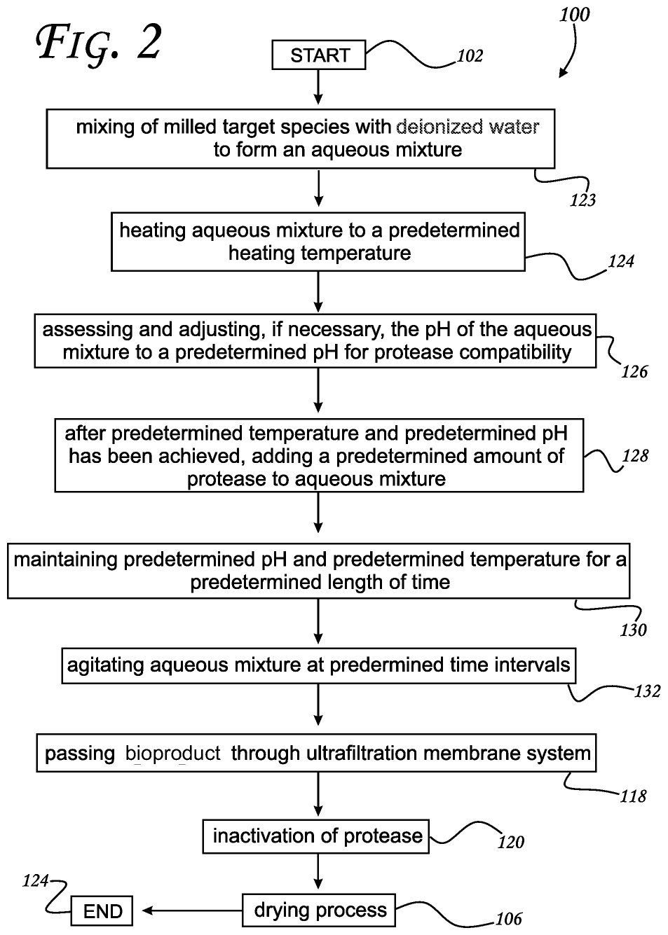

Illustrated in FIG. 2 is a simple flow chart that may further elaborate on the hydrolysis process itself. The start 102 of the flow chart of FIG. 2 picks up just after the milling process 116 illustrated in FIG. 1. Accordingly, the milled material (not shown) may be mixed with deionized water 123 of a predetermined amount for form an aqueous mixture (described in FIG. 2 but not shown). The amount of deionized water mixed with milled sea cucumber may be of any amount desired for optimal outcome and is further elaborated on in later sections of this application. After the milled sea cucumber is mixed with deionized water 123, the aqueous mixture may then be heated up in a heating process 124 which may raise the temperature to a level wherein the greatest enzymatic activity may be achieved. There may be variance in pH levels between batches of aqueous mixtures, thus there may exist a need to perform a pH assessment 126 on the aqueous mixture to ensure that the solution is not too acidic or basic.

Still referring to FIG. 2, when the aqueous mixture is fully ready to be hydrolyzed, a predetermined amount of a protease addition 124 may be applied to the aqueous mixture to catabolize proteins located in the aqueous mixture. The aqueous mixture may need to have full maintenance of heat 130 at a predetermined temperature and pH for a duration of time to fully catabolize the protein content of the aqueous mixture. Routine agitating 132 may likewise be useful in this process. After the maintenance of heat 130 for a predetermined time is complete, the aqueous mixture may be passed through the previously described ultra-filtration membrane system 118 to separate the hydrolysate (not shown) from the aqueous mixture. The hydrolysate may contain protease and may still be in an aqueous state. Thus, an inactivation process 120 may be needed to halt enzymatic activity. Once the inactivation process 120 is complete, the drying process 122 may proceed. Other steps may be performed, and FIG. 2 is not fully inclusive nor fully exclusive of all possible steps.

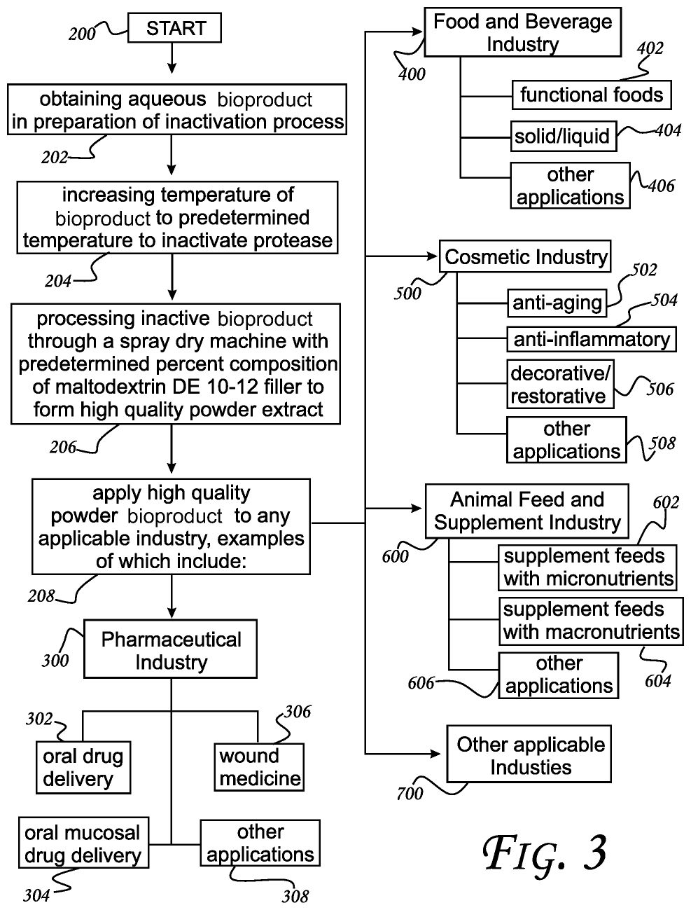

FIG. 3 illustrates, by way of a simple flow chart, the inactivation process 120 and drying process 122 of FIGS. 1 and 2 in greater detail. Additionally, FIG. 3 may be illustrative of the various industries that finished high-quality sea cucumber bioproduct may be applied towards. Accordingly, the inactivation process 120 (shown in FIG. 2) of FIG. 3 may be illustrative of the sub-steps for obtaining aqueous hydrolysate 202 and applying an additional increase in temperature 204 over the maintenance of heat 130 as described in FIG. 2. By employing an additional increase in temperature 204 to the active protease enzymes, the protease will denature and become inactive as described in the inactivation process 120 of FIGS. 1 and 2. After the inactivation process 120 (as shown in FIG. 2) is complete the drying process 122 (as shown in FIG. 2) may begin. Accordingly, FIG. 3 shows that the drying process 122 may occur by processing inactive hydrolysate 206 through a spray machine with a predetermined amount of maltodextrin DE 10-12 filler. Using a spray machine as described in FIG. 3 may result in a high-quality powder bioproduct 208. The high-quality powder bioproduct 208 may be employed in a wide variety of industries, including by not limited to; the pharmaceutical industry 300, the food and beverage industry 400, the cosmetic industry 500, the animal feed and supplement industry 600, and other industries 700 not herein described.

FIG. 3 further illustrates that the pharmaceutical industry 300 may have specific methods of drug delivery which are more readily apparent such as oral drug delivery 302, oral mucosa drug delivery 304, and wound medicine 306. However, these are just the most immediately apparent targets of the high-quality powder bioproduct 208. There may be other pharmaceutical uses 308 which may be equally suitable for targeted uses of the high-quality powder bioproduct 208 and failure to mention these uses in this disclosure does not limit this disclosure to only the uses mentioned herein. Accordingly, additional pharmaceutical industry 300 uses may be described later in this disclosure.

FIG. 3 further illustrates that the food and beverage industry 400 may have specific methods of use which are more readily apparent such as functional foods 402 and both solid and liquid foods 404 for example, energy drinks. However, these are just the most immediately apparent targets of the high-quality powder bioproduct 208. There may be other food and beverage industry 400 uses which may be equally suitable for the high-quality powder bioproduct 208 and failure to mention these uses in this disclosure does not limit this disclosure to only the uses mentioned herein. Accordingly, additional food and beverage industry 400 uses may be described later in this disclosure.

FIG. 3 further illustrates that the cosmetic industry 500 may have specific methods of use which are more readily apparent such as anti-aging 502 applications and anti-inflammatory 504 applications. However, these are just the most immediately apparent targets of the high-quality powder bioproduct 208. There may be other cosmetic industry 500 uses which may be equally suitable for the high-quality powder bioproduct 208 and failure to mention these uses in this disclosure does not limit this disclosure to only the uses mentioned herein. Accordingly, additional cosmetic industry 500 uses may be described later in this disclosure. Other steps may be performed, and the FIG. 3 is not fully inclusive nor fully exclusive of all possible steps.

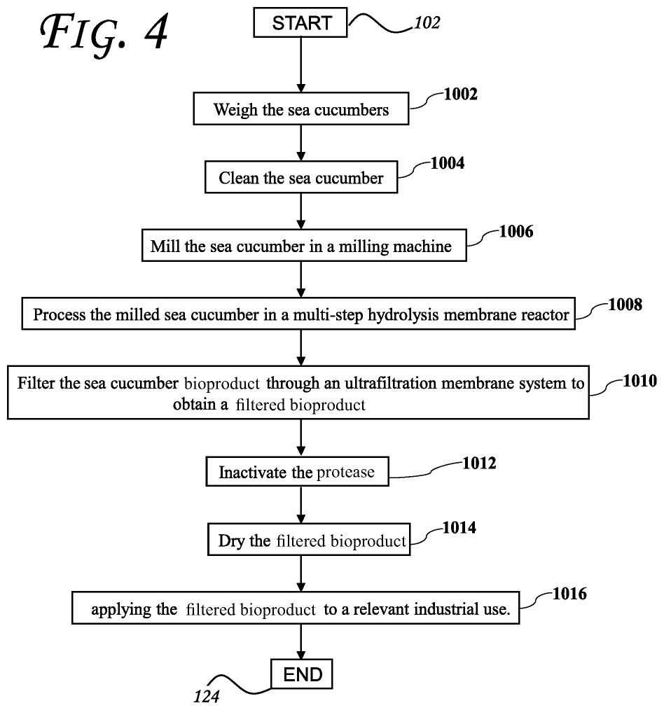

FIGS. 4, 5, 6, 7, 8A, and 8B, are further additional simplified flow charts diagramming various embodiments of this present disclosure. FIG. 4 may describe a method of producing a hydrolysate bioproduct from a sea cucumber wherein a sea cucumber is weighted 1002, cleaned 1004, milled in a milling machine 1006, processed the in a multi-step hydrolysis membrane reactor for creating a hydrolyzed sea cucumber 1008, filtered through an ultrafiltration membrane system to obtain a purified hydrolysate bioproduct 1010, inactivated to halt protease activity 1012, dried 1014, and applied to a relevant industrial use 1016. Other steps may be performed, and FIG. 4 is not fully inclusive nor fully exclusive of all possible steps.

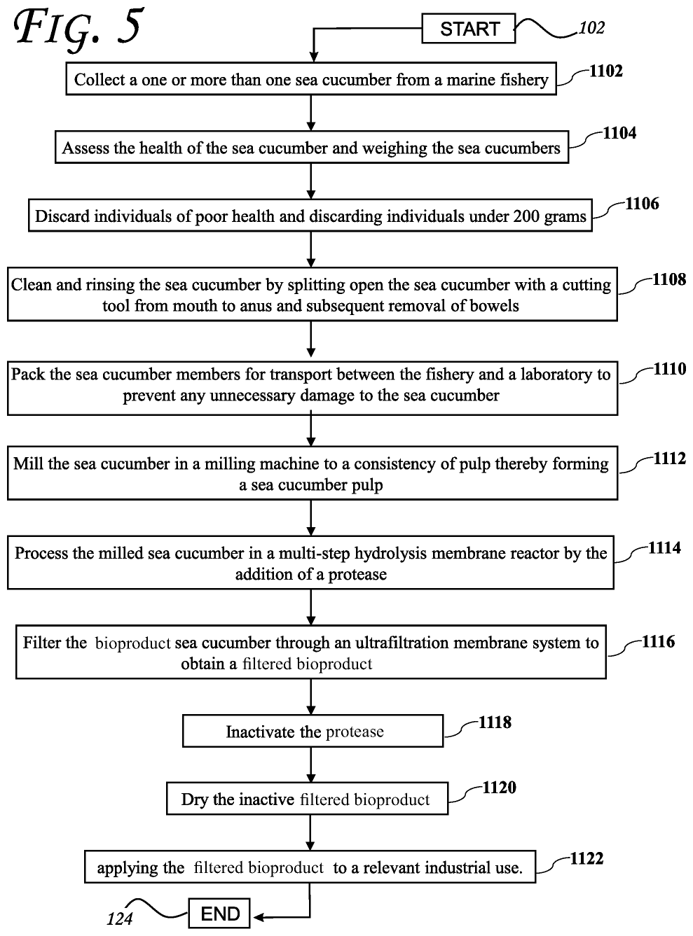

FIG. 5 may describe a method of producing a hydrolysate bioproduct from a sea cucumber wherein the following steps are described; the collecting a one or more than one sea cucumber from a marine fishery 1102 may be performed, the assessment of the health of the sea cucumber and weighing the sea cucumbers 1104 may occur, then discarding individuals of the poor health and discarding individuals under 200 grams 1106 due to quality control and sustainability concerns, followed by the cleaning and rinsing of the sea cucumber by splitting open the sea cucumber with a cutting tool from mouth to anus and subsequent removal of bowels 1108. After an appropriate harvest, the sea cucumber may need to be packaged for transport between the marine fishery and a laboratory to prevent any unnecessary damage to the sea cucumber 1110. Once back at a laboratory, the sea cucumber may be milled in a milling machine to a consistency of pulp thereby forming a sea cucumber pulp 1112. The sea cucumber pulp may be processed in a multi-step hydrolysis membrane reactor by the addition of a protease 1114. Filtering the hydrolyzed sea cucumber through an ultrafiltration membrane system to obtain a purified hydrolysate bioproduct 1116 follows. Inactivating the protease 1118 may follow the filtering step. Then a drying step may need to occur to the hydrolysate bioproduct 1120, and finally, the hydrolysate bioproduct may be ready to be applied to a relevant industrial use 1122. Other steps may be performed, and the FIG. 5 is not fully inclusive nor fully exclusive of all possible steps.

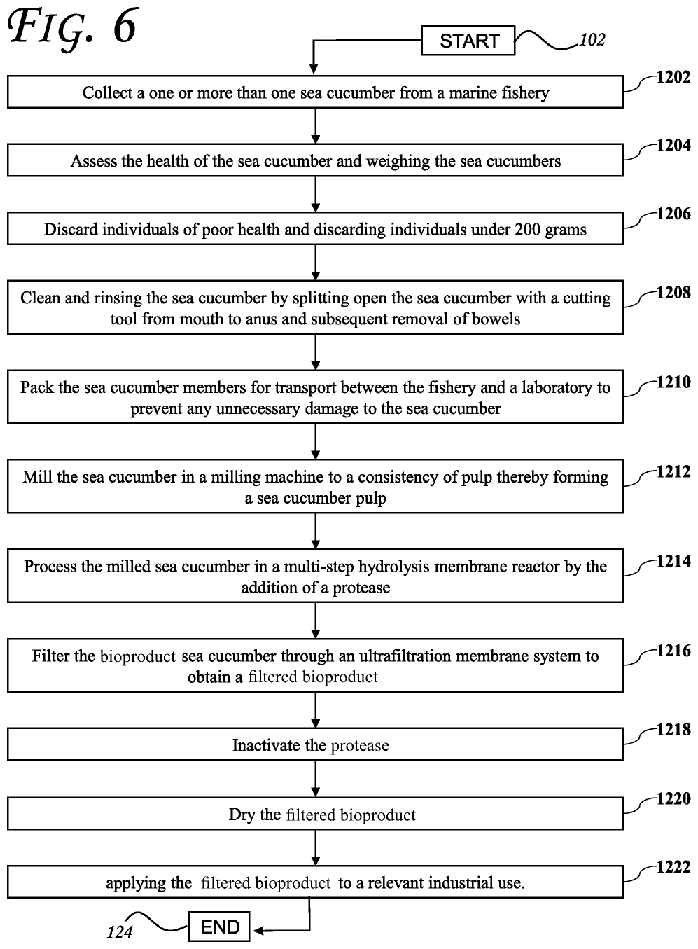

FIG. 6 may describe a method of producing a hydrolysate bioproduct from a sea cucumber wherein the following steps are described; the collecting a one or more than one sea cucumber from a marine fishery 1202 may be performed, the assessment of the health of the sea cucumber and weighing the sea cucumbers 1204 may occur, then discarding individuals of the poor health and discarding individuals under 200 grams 1206 due to quality control and sustainability concerns, followed by the cleaning and rinsing of the sea cucumber by splitting open the sea cucumber with a cutting tool from mouth to anus and subsequent removal of bowels 1208. After an appropriate harvest, the sea cucumber may need to be packaged for transport between the marine fishery and a laboratory to prevent any unnecessary damage to the sea cucumber 1210. Once back at a laboratory, the sea cucumber may be milled in a milling machine to a consistency of pulp thereby forming a sea cucumber pulp 1212. The sea cucumber pulp may be processed in a multi-step hydrolysis membrane reactor by the addition of a protease 1214. Filtering the hydrolyzed sea cucumber through an ultrafiltration membrane system to obtain a purified hydrolysate bioproduct 1216 follows. Inactivating the protease 1218 may follow the filtering step. Then a drying step may need to occur to the hydrolysate bioproduct 1220, and finally, the hydrolysate bioproduct may be ready to be applied to a relevant industrial use 1222. Other steps may be performed, and FIG. 6 is not fully inclusive nor fully exclusive of all possible steps.

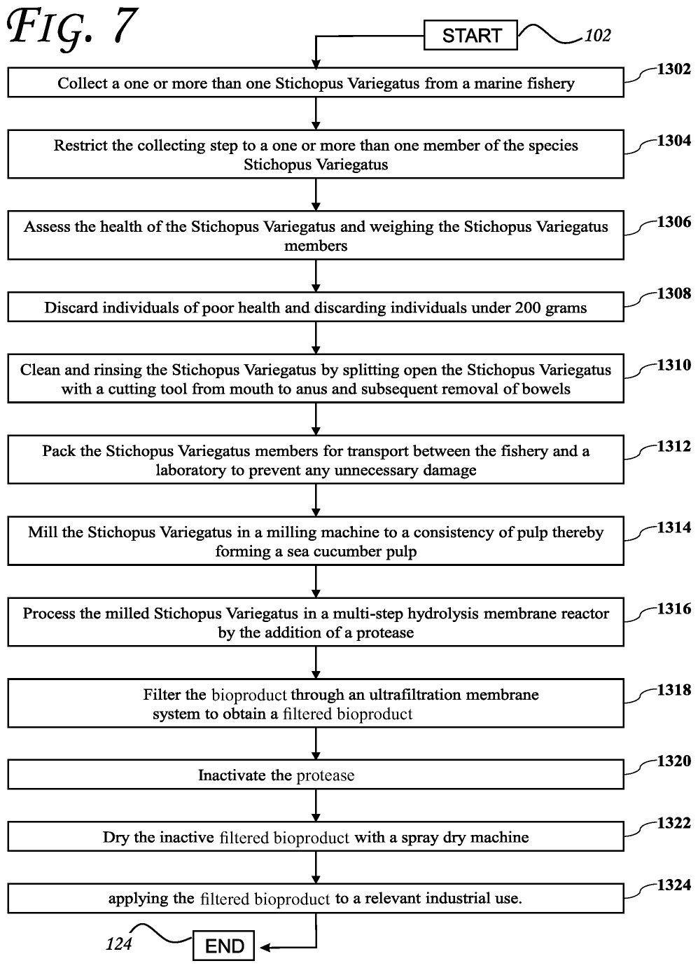

FIG. 7 may describe a method of producing a hydrolysate bioproduct from a sea cucumber wherein the following steps are described; collecting a one or more than one sea cucumber of the species Stichopus variegatus from a marine fishery 1302 may be performed. The collection step may utilize a sub-step wherein the collection is restricted to the species Stichopus variegatus 1304. The assessment of the health of the sea cucumber of the species Stichopus variegatus and weighing the sea cucumbers 1306 may occur, then discarding individuals of the poor health and discarding individuals under 200 grams 1308 due to quality control and sustainability concerns, followed by the cleaning and rinsing of the species Stichopus variegatus sea cucumber by splitting open the sea cucumber with a cutting tool from mouth to anus and subsequent removal of bowels 1310. After an appropriate harvest, the sea cucumber may need to be packaged for transport between the marine fishery and a laboratory to prevent any unnecessary damage to the sea cucumber 1312. Once back at a laboratory, the sea cucumber may be milled in a milling machine to a consistency of pulp thereby forming a sea cucumber pulp 1314. The sea cucumber pulp may be processed in a multi-step hydrolysis membrane reactor by the addition of a protease 1316. Filtering the hydrolyzed sea cucumber through an ultrafiltration membrane system to obtain a purified hydrolysate bioproduct 1318 follows. Inactivating the protease 1320 may follow the filtering step. Then a drying step may need to occur to inactive the hydrolysate bioproduct 1322, and finally, the hydrolysate bioproduct may be ready to be applied to a relevant industrial use 1324. Other steps may be performed, and the FIG. 7 is not fully inclusive nor fully exclusive of all possible steps.

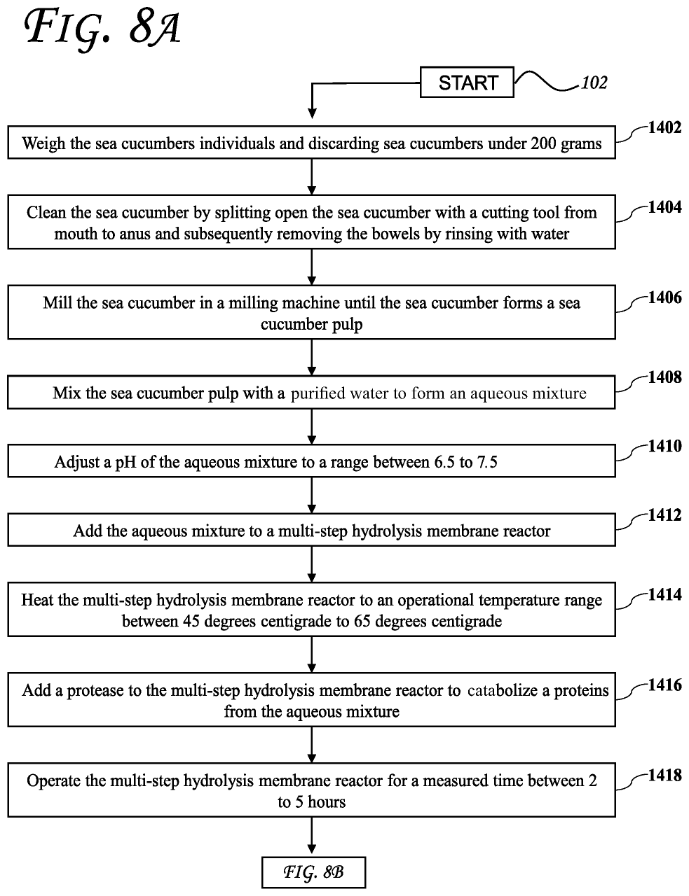

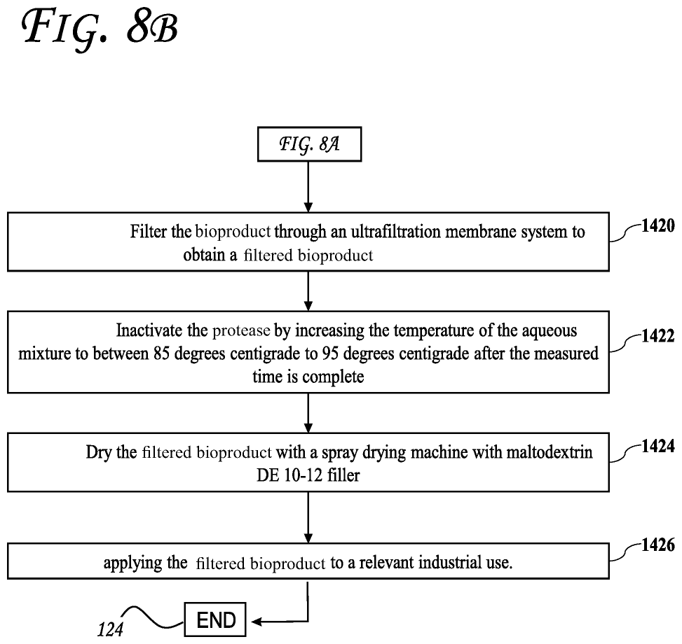

FIGS. 8A and 8B may describe a method of producing a hydrolysate bioproduct from a sea cucumber wherein the following steps are described; weighing the sea cucumbers may occur and discard individuals under 200 grams 1402, followed by the cleaning and rinsing of the sea cucumber by splitting open the sea cucumber with a cutting tool from mouth to anus and subsequent removal of bowels 1404. At a laboratory, the sea cucumber may be milled in a milling machine to a consistency of pulp thereby forming a sea cucumber pulp 1406. The sea cucumber pulp may be mixed with deionized water for form an aqueous mixture 1408. To ensure proper enzymatic function, a pH check and adjustment may be needed 1410. The sea cucumber pulp may be added to a multi-step hydrolysis membrane reactor 1412. The temperature of the multi-step membrane reactor may be increased to a range between 45 to 65 degrees centigrade 1414. The addition of a protease may then take place to catabolize the protein content of the aqueous mixture 1416. The multi-step membrane reactor may be operated for between 2 to 5 hours 1418. FIG. 8B then shows a filtering step for the hydrolyzed sea cucumber through an ultrafiltration membrane system to obtain a purified hydrolysate bioproduct 1420. Inactivating the protease may follow the filtering step by increasing the temperature to between 85 to 95 degrees centigrade 1422. Then a drying step may need to occur to the hydrolysate bioproduct using maltodextrin DE 10-12 1424, and finally, the hydrolysate bioproduct may be ready to be applied to a relevant industrial use 1426. Other steps may be performed, and FIGS. 8A and 8B are not fully inclusive nor fully exclusive of all possible steps.

Having disclosed the structure, form, and methods of use of the preferred embodiments, it is now possible to describe its function, operation, and use.

Sea cucumbers typically have soft, quadrangular bodies and may be covered on all four sides with thick tubercles. The species, Stichopus variegatus, may be covered in small dark spots and may be found in reefs and sand flats alike. The mean weight may be between 1000 to 2500 grams with a body wall thickness of approximately 0.8 centimeters. Said body wall may easily disintegrate outside of seawater and may be relatively large or range from medium to large. The distribution of Stichopus variegatus is considered to be Indo-Pacific with the exclusion of Hawaii. The Stichopus variegatus species is rarely collected due to the common depth of the species, and because the tegument disintegrates very easily which together, may result in low commercial value of the species. Accordingly, the low commercial value of the species may result in a reduced understanding of the various pharmacologic, cosmetic, and nutritional uses which the species may be employed for. This disclosure seeks to address at least some of these shortcomings.

Several unique biological and pharmacological activities including anti-angiogenic, anticancer, anticoagulant, anti-hypertension, anti-inflammatory, antimicrobial, antioxidant, antithrombotic, antitumor and wound healing have been ascribed to various species of sea cucumbers. Therapeutic properties and medicinal benefits of sea cucumbers may be linked to the presence of a wide array of bioactive compounds, especially triterpene glycosides (saponins), chondroitin sulfates, glycosaminoglycan (GAGs), sulfated polysaccharides, sterols (glycosides and sulfates), phenolics, cerebrosides, lectins, peptides, glycoprotein, glycosphingolipids, and essential fatty acids.

Bioproduct as used herein may be defined as a collection of active compounds, which may include frondoside A, of the specific sea cucumber species identified from the Stichopus variegatus species of sea cucumber. The disclosed bioproduct may be a unique new product manufactured from sea cucumber Stichopus variegatus species. The enzymatic process may produce a sea cucumber hydrolysate bioproduct with a small particle size. The bioproduct may have an exceptional nutritional value and may contain all components necessary for collagen synthesis and support of vital metabolic functions.

Bioproduct may further be a collection of active ingredients which can be hydrolyzed from sea cucumbers using a protease enzyme such as Alcalase®. Hydrolysate may refer to any product of hydrolysis. Hydrolysis may be described as a process of decomposition of proteins with the addition of proteolytic enzymes with the final result being a mixture of protein components. Proteolytic enzymes cause proteolysis, which may be described as the breakdown of proteins into smaller polypeptides or amino acids. In general, the hydrolysis process may be carried out on a wide variety of marine species to break down proteins into constituent amino acids. Sea cucumber hydrolysis may be performed in an effort to maximize the total yield bioproduct of sea cucumbers.

Tables of possible compositions and analysis of bioproduct product. Such below values are by approximation only.

| | |
|---|---|
| Collagen content approximate value | 85% |
| Typical profile of amino acid (method: 18517/MU/UPLC) | |
| Amino acid content (mg/Kg) | |
| Alanine | 4909 |
| Arginine | 3842 |
| Aspartic acid & Asparagine | 5128 |
| Glutamic acid & Glutamine | 8663 |
| Glycine | 9182 |
| Histidine | 501 |
| Hydroxyproline | Na |
| Isoleucine | 1540 |
| leucine | 4145 |
| Lysine | 2239 |
| Methionine | Na 739 |
| Phenylalanine | |
| Proline | 4479 |
| Serine | 2613 |
| Threonine | 3123 |
| Thyroxin(mg/Kg) | Na |
| Tryptophan | Na |
| Tyrosine | 918 |
| Taurine | Na |
| Valine | 2287 |

### Important Micronutrients

| | Amount | Unit |
|---|---|---|
| Manganese (Mn) | 1162 | mcg/Kg |
| Zinc (Zn) | 2.12 | mg/Kg |
| Magnesium (Mg) | 215 | mcg/Kg |
| Vitamin C | <0.13 | mg/Kg |
| Vitamin D | <0.67 | mg/Kg |

### Other Product Characteristics

| | Value | Unit |
|---|---|---|
| Protein | >90 | % |
| pH | 7-8 | pH |
| Sulfites | <2 | % |
| Typical Molecular Weight (Mw) | <2000 | Da mg/K g |
| Glucosamine | 325951.1 | |

Dried sea cucumbers may have a high nutrient content that is rich in protein, unsaturated fatty acids, minerals, vitamin B complex, and several bioactive compounds. Examples of some unsaturated fatty acids may include omega 3. Examples of minerals may include magnesium, phosphorus, sodium, potassium, zinc, and copper. Examples of vitamin B complexes may include thiamine, riboflavin, and niacin. Examples of bioactive compounds may include lectins, glucosamine, chondroitin sulfate, mucopolysaccharides, and glycoside saponins. Any single nutrient of a composition of hydrolysate bioproduct may be purified and isolated for use in any one of the various industries identified in this disclosure.

As noted, sea cucumbers may have a high protein content, at times approximately 72% by weight. Much of that protein content is applicable to the pharmaceutical and medical industries. Approximately 70% of the total composition of the protein may be of a type of identified in the formation of collagen. The benefits of collagen in the body include antiaging, maintenance of good joint and bone health, and having capabilities to accelerate wound healing.

Additionally, omega 3 fatty acids may have the capability to reduce triglyceride and cholesterol levels in the blood, accelerate wound healing, and inhibit prostaglandin formation and vasodilation resulting from activation of the inflammation pathway.

Further, lectins from sea cucumber extract may inhibit the growth of cancer cells and may have a positive effect on the resistance of the HIV virus. Glucosamine may boost immune system function, prevent joint problems, reduce inflammation or act as an anti-inflammatory, and also may reduce the risk of atherosclerosis. Chondroitin sulfate may have the benefit of preventing joint loss, repairing cartilage tissue, and when used as a supplement may increase stamina. Saponin glycosides may have the same chemical structure as Ganoderma compounds found in sea ginseng. This compound is reported to be able to inhibit the growth of cancer cells and may function as a nutritional supplement tonic.

Collagen may be considered a type of protein that largely forms connective tissue. Such connective tissue may be identified as a form of fibrous tissue that makes up about one-third of animal vertebrate bodies. Collagen is the main component of teeth, meat, bone, and skin layers in mammals. Collagen may be the most abundant protein of both vertebrate and invertebrate bodies. Collagen may be composed of unique amino acids that form the structure of a triple helix. Moreover, glycine is the main amino acid constituting collagen and may always be located in the third position of each repetition of an amino acid sequence. The main structure of collagen, therefore, may be gly-x-y, where x may usually contain the amino acid proline while y may contain the amino acid hydroxyproline.

Hydroxyproline may be a proline derivative of post-translational hydroxylation reaction mediated by prolyl hydroxylase. Further, hydroxyproline is considered a major component of the protein collagen and may play a role in the stability of the collagen triple helix. Collagen also contains an unusual amino acid, hydroxylysine. Hydroxylysine may be formed from lysine which is hydroxylated by the enzyme lysyl hydroxylase. Both of these amino acids may form stable hydrogen bonds and structures from the triple-helical collagen.

Bioactive peptides may be considered as pieces of specific proteins that may have a positive effect on the body and may affect overall health. Accordingly, protein in the intact form may have relatively low bioactivity. Proteins that have been hydrolyzed with enzymes may subsequently increase in overall bioactivity due to the protein being released from a long bond and fragmented. Bioactive peptides may have potential as antihypertensive compounds, antioxidants, opioid antagonists, antibacterial, antithrombotic, and immunomodulators. Peptides produced from food proteins can lower blood pressure, maintain body weight balance, inhibit prolin specific endopeptidase activity, enhance the immune system, inhibit blood platelet aggregation, inhibit HIV proteinase and oxidation processes, may have antibacterial and antiviral activity, bind ions and help transport minerals and improve the nutritional value of food.

Bioactive peptides may be produced in several ways, namely through enzymatic hydrolysis with digestive enzymes, fermentation processes using microbial activity, and chemical synthesis. Enzymatic hydrolysis of proteins with suitable proteolytic enzymes may be capable of producing peptides with expected activity. Physio-chemical conditions of the substrate such as temperature 124 and pH 126 of the solution may be required to be in accordance with the optimal operative conditions of the enzyme for proper enzymatic function. Some of the enzymes commonly used for hydrolysis include papain, trypsin, α-chymotrypsin, pepsin, bromelain, Alcalase®, and neutrase.

Accordingly, one of the more important factors in peptide production of bioactives may be the molecular weight of the starting peptide. A method commonly used to produce peptides with certain molecular weights may be the ultrafiltration membrane system 118. Tiered hydrolysis system may function in principal by utilizing several enzymes that may be capable of producing peptides with smaller sizes. The combination of the multistep hydrolysis membrane reactor system 116 and the ultrafiltration membrane system 118 may be capable of producing peptides with optimal activity and may be utilized herein.

Hydrolysate may be defined as the product of the hydrolysis process. Hydrolysis may be described as the breakdown of larger proteins into constituent amino acids. Bioproduct may be the end product of hydrolysis. The object of this disclosure may be the process of sea cucumber hydrolysis using a protease enzyme such as Alcalase® to produce bioactive bioproducts found in sea cucumbers, especially collagen, glucosamine, amino acids, and other bioproducts.

Sea cucumber bioproduct may be produced as a powder, may be used for raw materials and preparations for the pharmaceutical industry, the cosmetics industry, the food and beverage industry, the animal feed industry, and other industries and their use.

The stages of making bioproduct may be generally described as follows:
Selecting and obtaining (by physical lift from a benthic environment) the freshest selection of sea cucumber 104, specifically the species Stichopus variegatus, often with the use of professional divers who utilize mixed gases in a breathing apparatus
Weighing individuals and selecting those above 200 grams per animal 108 for sustainability reasons.
Assessing the health and viability of the species 106.
Discarding individuals who display poor health.
Cleaning and rinsing the sea cucumber by removal of bowels 110.
Handling of sea cucumber between any given fishery and the laboratory to prevent any unnecessary damage to the sea cucumber 112.
Milling the sea cucumber at a laboratory 114.
Obtain bioproduct by hydrolysis process through the use of the multistep hydrolysis membrane reactor system 116 and the ultrafiltration membrane system 118.
Inactivation process 120.
Drying process 122.

Sea cucumbers used in the process of making bioproduct may be sea cucumbers of the Holothuroidea class. The selection process 106 of sea cucumber restricts the use and selection of sea cucumber to a weight above 200 per gram 108. Fresh sea cucumbers are then taken from the fishery 104 and cleaned 110 such that the contents of the stomach are emptied by splitting the sea cucumber from the back of the mouth to the sea cucumber anus. Sea cucumbers are cleaned and rinsed using clean water to remove dirt and minerals 110.

Of particular note, the harvest of sea cucumbers individuals may be restricted to individuals weighing more than 200 grams. Such restriction may be by choice due to sustainability concerns. However, where populations are noted to be in abundance, individuals under 200 grams may likewise be harvested, and the 200 gram weight limit shall not be understood to be a limitation herein. All methods described herein may occur with any weight individual sea cucumber.

The milling process 114 of sea cucumber may utilize a grinding machine. The grinding machine mills the sea cucumber until the tissues break and the appearance resembles that of pulp. Pulp as used herein shall refer to a soft, wet, shapeless mass of material. The particle size of sea cucumber pulp will affect the hydrolysis process, and accordingly, the smaller the particle size the sea cucumber may be broken down to the faster the hydrolysis process will occur. There is no required particle size of the pulp, rather it shall be generally understood that smaller particle size is preferred but any particle size of the pulp may be hydrolyzed into bioproduct.

The hydrolysis process as described in FIG. 2 may be carried out using a percentage of 40% sea cucumbers and 60% purified water to produce the most optimum bioactive content 123. Purified water in this situation may refer to de-ionized water, filtered water, distilled water, or water that has gone through a reverse osmosis filtration system. The hydrolysis process may be carried out at a temperature of 45-65 degrees centigrade 124 and at an approximately neutral pH of around 6.5-7.5 126. After the appropriate temperature is achieved, a 2% concentration of a protease may be added to the solution for enzymatic breakdown 128. Protease enzymes will be active at temperatures ranging from 35-65 degrees centigrade, and may optimally function at temperatures between 50-55 degrees centigrade 130. The hydrolysis process may be carried out for two to five hours from the time the enzyme is added 130 and may be routinely agitated 132 during this process.

After the hydrolysis process is completed, then the inactivation process 120 is continued for anywhere between ten and twenty minutes at temperatures of 85-95 degrees centigrade 204. The inactivation process 120 is carried out to halt all enzyme activity 204. The sea cucumber hydrolysis process up to this point has occurred in a liquid medium and therefore is required to be dried prior to being useable.

The drying process 106 may utilize a dry spray machine 206. Such spray dry machine may be ideal for maintaining maximum bioactive content of sea cucumber hydrolysis and thereby may produce a high-quality powder bioproduct. The drying process may use maltodextrin DE 10-12 fillers 206. Such fillers may constitute as much as 5-30% by weight or by volume of dried bioproduct.

As noted, the sea cucumber hydrolysate bioproduct preparations may contain active ingredients that may prove to be useful in pharmaceuticals 300, cosmetics 500, and functional foods 400. The content of active sea cucumber ingredients such as chondroitin sulfate, glucosamine, minerals, and frondoside A, may be very beneficial in the world of health, nutrition, beauty, and pharmaceuticals.

The search for anti-cancer compounds from different marine animals has revealed thousands of active compounds. Echinoderms are one of the marine phylum which includes sea stars, sea urchins, sea cucumbers, and sea lilies. Sea cucumbers have been widely used in traditional Chinese medicine for the treatment of cancer, inflammation, and other health cures for hundreds of years.

Terpenoid glycosides from various species of sea cucumbers are known to have anti-cancer activity. One of the special triterpenoid glycosides produced by sea cucumbers is frondoside A, which has received attention in the pharmaceutical world. Sea cucumbers have shown strong anti-cancer effects in cancer malignancies, one of which is leukemia. As stated, the term bioproduct may be used herein to describe the active compound of frondoside A, along with other active compounds, of the specific sea cucumber species identified as Stichopus variegatus.

Frondoside A may be a triterpenoid glycoside with an acetoxy group on C-16 in aglycones, which is a derivative of lanostane. Frondoside A is pentaoside with xylose as the third monosaccharide residue and 3-O-methylglucose as the terminal monosaccharide residue. Frondoside A has a molecular mass of 1334 Da. There are 3 types of frondoside namely frondoside A, B, and C. Frondoside (A, B, and C) can be easily isolated and purified, compounds that are proven to have the highest purity, namely frondoside A. Research suggests that bioproduct from sea cucumbers contain terpenoid glycosides as anti-cancer agents, so sea cucumber hydrolysate bioproduct may be effective in anticancer treatment.

The common method for extraction of Frondoside A, as learned from other scientific articles, is performed from either the freeze-dried cooking water from the sea cucumber processing plant or freeze-dried skin of the animal. The method described herein may be considered novel and more efficient at obtaining higher quantities of Frondoside A, and specifically, bioproduct than the aforementioned common methods. Typically, the freeze-dried powders would be dissolved in chloroform/methanol and evaporated. Following evaporation, the extract would have been dissolved in water and mixed with ethyl acetate. After phase separation, the water phase may then have been loaded onto a Teflon column. The column would then have been washed with water to remove salts and pigments and the crude glycoside fraction then eluted with 65% acetone. The glycosides would then have been purified on a Si 40 L 2632-2 flash column with the mobile phase mixture of chloroform/ethanol/water (100:100:17) used as a solvent. Purification would be monitored by thin-layer chromatography with 100:100:17 chloroform/ethanol/water as the solvent system. The Frondoside A yield may have been approximately 0.1% of either starting material. Acridly, by utilizing a hydrolysis process described herein, much higher volumes of Frondoside A, and specifically, overall bioproduct may be obtained.

Sea cucumber bioproduct preparations may have applicability in the pharmaceutical, cosmetics, food and beverage, animal feed, and other industries. These various industries will be reviewed in tern below.

A) Application in pharmacy. Pharmaceutical industry 300 uses.
1. Oral Drug Delivery 302 of bioproduct.
   a. Sea cucumber bioproduct material preparations in powder form may be administered in capsule or tablet form. In capsule preparations, the use 300 mg, 500 mg and 600 mg capsules may be prescribed.
2. Oral Mucosal Drug Delivery 304 of bioproduct.
   a. Sea cucumber bioproduct preparations may be applied orally via lozenges with formulations with other additives as a base for lozenges.
3. Wound Medicine 306 application of bioproduct.
   a. Sea cucumbers contain active compounds which may serve as anti-inflammatory agents, and thus may be useful in accelerating wound healing. Sea cucumber bioproduct can be formulated as an external medicine liquid for external wounds and applied topically. Sea cucumbers also contain high collagen and thus may be very effective in skin regeneration.

B) Application in cosmetics. Cosmetic industry 500 uses.
1. Collagen as anti-aging 502 of bioproduct.
   a. The high collagen content in sea cucumbers bioproduct, up to 80%, may be very beneficial if applied as a topical supplement for skincare. The results of some studies have suggested that the content of sea cucumber collagen is higher than the collagen content of land animals by percent composition of the animal. Moreover, because the human body appears primed to more readily absorb collagen when sourced from sea cucumber than from a land mammal, use of sea cucumber collagen may prove to be more commercially efficient than sourcing collagen from land animals.
2. Collagen as anti-inflammatory 504 via application of bioproduct.
   a. Because sea cucumber bioproduct may contain numerous compounds that reduce inflammation, the applicability of a collagen-based anti-inflammatory product for inclusion in a cosmetic may, therefore, be for both the purpose of direct skincare and conditioning and may have a decorative based purpose.

C) Application in functional foods and drinks, food and beverage industry 400.
1. Solid foods 404 via application of Bioproduct™:
   a. A wide variety of solid foods may be developed and targeted for the addition of sea cucumber bioproducts. By way of example only, biscuits, crackers, bread, cookies, muffins, protein-based snacks, and many other foods that may already contain high protein content. Such additions may aid in further fortifying a wide variety of shelf-stable products.
   b. Additionally, where solid foods may be lacking in overall protein content, the addition of bioproduct to these protein-deficient foods may aid in bulking up the total protein content of the foods without the increase of excessive caloric content.
2. Beverage and semi-solid foods 404 via application of bioproduct:
   a. The high mineral and amino acid content makes sea cucumber bioproduct a very beneficial addition to beverages and may aid in further enhancing soft foods. Both beverages and semi-soft/solid foods may be turned into functional foods by the addition of raw material obtained from sea cucumber bioproduct. One potential example may be the application of bioproduct in energy drinks.
   b. Functional foods 402. Further, it may now be possible to increase the protein content of beverages as an analog to the discussion related to solid foods. While many beverages currently are touted to have high protein content, these types of beverages also tend to be high in calorie count as well. The use of sea cucumber bioproduct may increase the protein content of a beverage with a minimal impact on the total calorie content of a beverage.

D) Application of bioproduct in animal feed. Animal feed and supplement industry 600.
1. Animal feeds, such as fish, chicken, birds require a balanced intake of macronutrients and micronutrients. One example of a macronutrient is that of protein. Because sea cucumber bioproduct has very high protein content, in addition to a wide array of micronutrients, the bioproduct may be applied in animal feed for the supplemental support and health of a wide variety of livestock.

The process of turning fresh sea cucumber into bioproduct may go through several stages which may include the collection of raw materials 104, selection of raw materials 106, the handling of raw materials 112, the hydrolysis process 128, the inactivation process 120, and the drying process 106. This listing of stages may not be exhaustive nor fully inclusive, nor is any listing of stages identified throughout this disclosure deemed to be exhaustive nor fully inclusive. The process of selecting raw materials 104 of fresh sea cucumbers may be limited to sea cucumbers with a size greater than 200 grams per head 108. The process may next proceed to where fresh sea cucumbers may be cleaned and the bowels removed 110, then milled until smooth 114. Subsequently, the hydrolysis process may use sea cucumbers 40% to 70% by volume or by weight, and purified water, may be added at a volume of 50% to 60% with a protease, such as Alcalase® enzyme, at concentrations of approximately 0.5 to 5% as described in FIG. 2. The hydrolysis process may be carried out at a temperature of 45 degrees centigrade to 65 degrees centigrade for 2 to 5 hours 130. After the hydrolysis process is complete, then the inactivation process 120 may be continued for 10 to 20 minutes at a temperature of 85 degrees centigrade to 95 degrees centigrade 204. This inactivation process 106 may be carried out in an effort to halt enzymatic activity. Sea cucumber hydrolysis is generally carried out in aqueous solution, thus a drying process 106 may follow. The drying process 106 of the sea cucumber bioproduct may use a dry spray method with fillers constituting 5% to 30% by volume or weight 206.

Sea cucumbers may be considered marine animals that contain bioproduct such as active ingredients for the benefits of anticancer properties, anticoagulant properties, antihypertensive properties, and may, therefore, be considered very effective in the treatment of cancer, cardiac, and hypertension treatments. Further, the content of collagen in sea cucumbers may reach 50-80% and may, therefore, be considered very useful when applied in the cosmetics field as an antiaging and anti-inflammatory as well. Active compounds in sea cucumbers such as proteins, minerals, and amino acids may also be very effective when applied in functional foods 402, including drinks, which are additionally considered functional foods 402. Such functional foods 402 developed from the active compounds of sea cucumber bioproduct can be targeted to pleasant snack foods such as, but not limited to, biscuits, chips, muffins, cookies, crisps, popcorn, crackers and other foods with low or high protein. Additionally, the active compound previously noted as being derived from sea cucumbers may also be applied in drinks to either boost the protein content or to supplement an already high protein content beverage. Such beverages may include but are not limited to, milk, energy drinks, coffee, and other drinks.

Based on the results of research that have been done stating that sea cucumber hydrolysate bioproduct contains terpenoid glycosides, also known as frondoside A, which is a known as an anti-cancer agent, production of sea cucumber hydrolysate bioproduct in a manner herein described may result in a very effective anticancer treatment. Each of the processes, steps, and more broadly this disclosure generally, wherein the hydrolysate bioproduct is produced, may be applied as raw material and preparation for the pharmaceutical industry 300, the cosmetics industry 500, the food and beverage industry 400, the animal feed industry 600, and other industries 700 and the use thereof.

While embodiments of the present disclosure have been illustrated and described, it is not intended that these embodiments illustrate and describe all possible forms of this disclosure. Rather, the words used in the specification are words of description rather than limitation, and it is understood that various changes may be made without departing from the spirit and scope of the disclosure.

Accordingly, it is not intended that the various embodiments be limited except by the appended claims. Insofar as the description above and the accompanying drawings disclose any additional subject matter that is not within the scope of the claims below, the embodiments are not dedicated to the public and the right to file one or more applications to claim such additional embodiments is reserved.

1: (canceled)

2: (canceled)

3: (canceled)

4: (canceled)

5: (canceled)

6: (canceled)

7: (canceled)

8: (canceled)

9: (canceled)

10: (canceled)

11: (canceled)

12: (canceled)

13: (canceled)

14: (canceled)

15: (canceled)

16: (canceled)

17: (canceled)

18: (canceled)

19: (canceled)

20: (canceled)

21: (canceled)

22: (canceled)

23: (canceled)

24: (canceled)

25: (canceled)

26: (canceled)

27: (canceled)

28: (canceled)

29: (canceled)

30: (canceled)

31: (canceled)

32: (canceled)

33: (canceled)

34: A method of producing a bioproduct from a sea cucumber species, Stichopus variegatus, comprising:
a collecting of a one or more than one sea cucumber from a marine fishery;
an assessing of a health of the sea cucumber and weighing the sea cucumbers;
a discarding of individuals of the poor health and under 200 grams;
a cleaning and a rinsing of the sea cucumber by a splitting open of the sea cucumber with a cutting tool from a mouth to an anus and a subsequent removal of a bowels;
a packing of the sea cucumber for transport between the marine fishery and a laboratory to prevent an unnecessary damage to the sea cucumber;
a milling of the sea cucumber in a milling machine to a consistency of pulp thereby forming a sea cucumber pulp;
a processing of the sea cucumber pulp in a multi-step hydrolysis membrane reactor by the addition of a protease to make an unfiltered bioproduct;
a filtering of the unfiltered bioproduct through an ultrafiltration membrane system to obtain a filtered bioproduct;
an inactivating of the protease;
a drying of the filtered bioproduct; and,
an applying of the filtered bioproduct to a relevant industrial use.

35: The method of producing a bioproduct from a sea cucumber species, Stichopus variegatus, of claim 34, wherein a purified water is combined with the sea cucumber pulp to form an aqueous mixture, the aqueous mixture being 60 weight percent purified water and 40 weight percent the sea cucumber pulp, and, the pH of the aqueous mixture is between 6.5 to 7.5.

36: (canceled)

37: The method of producing a bioproduct from a sea cucumber species, Stichopus variegatus, of claim 34, wherein the multi-step hydrolysis membrane reactor is operated at a temperature between 45 degrees centigrade to 65 degrees centigrade.

38: (canceled)

39: The method of producing a bioproduct from a sea cucumber species, Stichopus variegatus, of claim 34, wherein the aqueous mixture remains in the multi-step hydrolysis membrane reactor between 2 to 5 hours.

40: The method of producing a bioproduct from a sea cucumber species, Stichopus variegatus, of claim 34, wherein the protease in the aqueous mixture is inactivated by increasing the temperature of the aqueous mixture to between 85 degrees centigrade to 95 degrees centigrade.

41: (canceled)

42: The method of producing a bioproduct from a sea cucumber species, Stichopus variegatus, of claim 34, wherein the filtered bioproduct is dried by using a spray dry machine, the spray dry machine using a maltodextrin DE 10-12 filler, the maltodextrin DE 10-12 filler constituting between 5 weight percent and 30 weight percent of the filtered bioproduct.

43: (canceled)

44: The method of producing a bioproduct from a sea cucumber species, Stichopus variegatus, of claim 34, wherein the relevant industry is a pharmaceutical industry, a cosmetic industry, a food and beverage industry, or a livestock feed industry.

45: (canceled)

46: (canceled)

47: (canceled)

48: A method of producing a bioproduct from a sea cucumber species, Stichopus variegatus, comprising:
a collecting of a one or more than one Stichopus variegatus from a marine fishery;
a restricting of the collecting step to a one or more than one member of the species Stichopus variegatus;
an assessing of a health of the Stichopus variegatus and weighing the Stichopus variegatus;
a discarding of individuals of the poor health and under 200 grams;
a cleaning and a rinsing of the Stichopus variegatus by a splitting open of the Stichopus variegatus with a cutting tool from a mouth to an anus and subsequent removal of a bowels;
a packing of the Stichopus variegatus for transport between the marine fishery and a laboratory to prevent an unnecessary damage;
a milling of the Stichopus variegatus in a milling machine to a consistency of a pulp thereby forming a sea cucumber pulp;
a processing of the milled Stichopus variegatus in a multi-step hydrolysis membrane reactor by the addition of a protease to make an unfiltered bioproduct;
a filtering of the unfiltered bioproduct through an ultrafiltration membrane system to obtain a filtered bioproduct;
an inactivating of the protease;
a drying of the filtered bioproduct; and,
an applying of the filtered bioproduct to a relevant industrial use.

49: The method of producing a bioproduct from a sea cucumber species, Stichopus variegatus, of claim 48, wherein a purified water solution is combined with the sea cucumber pulp to form an aqueous mixture, the aqueous mixture being 60 weight percent purified water and 40 weight percent of the sea cucumber pulp and the pH of the aqueous mixture being between 6.5 to 7.5.

50: (canceled)

51: The method of producing a bioproduct from a sea cucumber species, Stichopus variegatus, of claim 48, wherein the multi-step hydrolysis membrane reactor is operated at a temperature between 45 degrees centigrade to 65 degrees centigrade.

52: (canceled)

53: The method of producing a bioproduct from a sea cucumber species, Stichopus variegatus, of claim 48, wherein the aqueous mixture remains in the multi-step hydrolysis membrane reactor for between 2 to 5 hours.

54: The method of producing a bioproduct from a sea cucumber species, Stichopus variegatus, of claim 48, wherein the protease in the aqueous mixture is inactivated by increasing the temperature of the aqueous mixture to between 85 degrees centigrade to 95 degrees centigrade.

55: The method of producing a bioproduct from a sea cucumber species, Stichopus variegatus, of claim 48, wherein the filtered bioproduct is dried by using a spray dry machine.

56: The method of producing a bioproduct from a sea cucumber species, Stichopus variegatus, of claim 48, wherein the spray dry machine uses a maltodextrin DE 10-12 filler, and the maltodextrin DE 10-12 filler is between 5 weight percent and 30 weight percent of the filtered bioproduct.

57: (canceled)

58: The method of producing a bioproduct from a sea cucumber species, Stichopus variegatus, of claim 48, wherein the relevant industry is a pharmaceutical industry, a cosmetic industry, a food and beverage industry, or a livestock feed industry.

59: (canceled)

60: (canceled)

61: (canceled)

62: A method of producing a bioproduct from a sea cucumber comprising:
a collecting of a one or more than one sea cucumber from a marine fishery;
a restricting of the collecting step to a one or more than one member of the species Stichopus variegatus;
an assessing of a health of the Stichopus variegatus and a discarding of unhealthy members;
a weighing of the sea cucumbers individuals and a second discarding of the sea cucumbers under 200 grams;
a cleaning of the sea cucumber by a splitting open of the sea cucumber with a cutting tool from a mouth to an anus and subsequently removing a bowels by rinsing with water;
a milling of the sea cucumber in a milling machine until the sea cucumber forms a sea cucumber pulp;
a mixing of the sea cucumber pulp with a purified water to form an aqueous mixture;
an adjusting of a pH of the aqueous mixture to between 6.5 to 7.5;
an adding of the aqueous mixture to a multi-step hydrolysis membrane reactor;
a heating of the multi-step hydrolysis membrane reactor to a temperature range between 45 degrees centigrade to 65 degrees centigrade;
an adding of a protease to the multi-step hydrolysis membrane reactor to catabolize a proteins from the aqueous mixture;
an operating of the multi-step hydrolysis membrane reactor for between 2 to 5 hours;
a filtering of the hydrolyzed sea cucumber through an ultrafiltration membrane system to obtain a filtered bioproduct;
an inactivating of the protease by an increasing the temperature of the filtered bioproduct to between 85 degrees centigrade to 95 degrees centigrade;
a drying of the filtered bioproduct with a spray drying machine with a maltodextrin DE 10-12 filler, the DE 10-12 filler between 5 weight percent and 30 weight percent of the filtered bioproduct; and,
an applying of the filtered bioproduct to a relevant industrial use.

63: The method of producing a bioproduct from a sea cucumber of claim 62, wherein the aqueous mixture is 60 weight percent of the purified water and 40 weight percent of the sea cucumber pulp.

64: (canceled)

65: The method of producing a bioproduct from a sea cucumber of claim 62, wherein the relevant industry is a pharmaceutical industry.

66: The method of producing a bioproduct from a sea cucumber of claim 62, wherein the relevant industry is a cosmetic industry.

67: The method of producing a bioproduct from a sea cucumber of claim 62, wherein the relevant industry is a food and beverage industry.

68: The method of producing a bioproduct from a sea cucumber of claim 62, wherein the relevant industry is a livestock feed industry.
